(19) United States
(12) Patent Application Publication
(10) Pub. No.: US 2016/0287179 A9
(43) Pub. Date: Oct. 6, 2016

(54) MULTIMEDIA TAGGING SYSTEM AND METHOD, RELATED COMPUTER PROGRAM PRODUCT

(71) Applicants: Alexandro SENTINELLI, Milano (IT); Marco PESSIONE, Milano (IT)

(72) Inventors: Alexandro SENTINELLI, Milano (IT); Marco PESSIONE, Milano (IT)

(73) Assignee: STMICROELECTRONICS S.R.L., Agrate Brianza (IT)

(21) Appl. No.: 13/616,916

(22) Filed: Sep. 14, 2012

(30) Foreign Application Priority Data
Sep. 15, 2011 (IT) TO2011A000823

(51) Int. Cl. G06Q 50/22

(52) U.S. Cl. CPC A61B 5/72; H04N 19/10

(57) ABSTRACT

An embodiment of a multimedia tagging system, includes a multimedia-content generator for producing multimedia content tagged with metadata, and a remote health-monitoring device for measuring and processing a set of biological and physiological signals of a user. The system is configured for tagging the multimedia content with tags extracted from a set of personal metadata obtained from the biological and physiological signals provided by the remote health-monitoring device and containing information relative to the emotional and health status of said user.

### PRIORITY CLAIM

The instant application claims priority to Italian Patent Application No. TO2011A000823, filed Sep. 15, 2011, which application is incorporated herein by reference in its entirety.

### TECHNICAL FIELD

An embodiment of the disclosure relates to semantic multimedia encoding. Various embodiments may relate to implementing automatic metadata tagging.

### BACKGROUND

Various documents disclose Multimedia Information Retrieval, or MIR. In this field a tag is produced while shooting a picture/video. The same tag may be possibly used to retrieve that content (usually stored as a file). This type of retrieve query is simply called “text query”.

Either through traditional text query tags (as used in search engines, such as “Google”), or via more sophisticated queries based on feature extraction, the retrieval may still be based on a simple matching of the query and the metadata of the content. The metadata is attached or extracted from a multimedia content, but is still linked to that particular data.

A secure Hash algorithm may be used to protect the integrity of the data, which has become popular with Torrent file-sharing applications. In a typical scenario, a user U1 first may produce a hash H1 that is a sort of a digital signature of the integrity of data D1. The user U1 sends to another user U2 the data D1 and the hash H1. After receiving the data D1, which is labeled D2 when received by the user U2, the user U2 may produce his own hash H2 and verify the correspondence with H1; if the correspondence is verified, D2 is held to be equal to D1. This verifies that the received data D2 is the same as the sent data D1.

Over the last twenty years or so, the evolution of microcomputers has had a huge impact on the development of medical instrumentation. In that area, the increased computing power and the capacity for such power to be compacted into relatively small chips make it possible to create “intelligent” devices that can be adapted to a specific patient. These devices may be able to monitor, detect, and recognize problems specific to a patient during the normal daily life. Wearable monitors can thus be developed thanks to improvements in reducing size, cost, and power consumption. With the availability of such wearable monitors that give low annoyance to the patient, it is possible to store the information on the patient and transmit it to a local hospital by using a telecommunication network. This improvement is beneficial both to the patient, as he or she can get back home as soon as possible, while being still monitored, and to the hospital, since money can be saved and beds made free for new patients.

Several studies have already been devoted to the estimation of how a patient “feels”, mainly with the aim of finding a correlation between the feeling status and the physiological indexes of the user.

A number of documents related to these studies will now be discussed. These documents will be referenced by a numeral between square brackets (i.e. [X]), the numeral referring to the list reproduced at the end of this description. All documents in this list are incorporated by reference.

Kim et al. [1] have developed an emotion-recognition system based on physiological signals, combining electrocardiogram, skin-temperature variation, and electrodermal-activity signals. After processing and feature extraction, through the use of a support-vector-machine classifier, such a system enables classifying four different emotion-specific characteristics.

Wagner et al. [2] proposed an emotion-recognition system that includes data analysis and classification of electromyogram, electrocardiogram, skin conductivity, and respiration changes, and uses a music-induction method, which elicits natural emotional reactions from the subject.

Goldstein at al. [3] performed a study where blood pressure (both systolic and diastolic) was correlated with different types of anger status.

Shapiro et al. [4] assessed the relationship between the intensity of single moods and mood combinations by measuring blood pressure and heart rate in nurses, and experienced graded increases in blood pressure and heart rate with higher ratings of negative moods, and decreases for a mood related to energy level.

### SUMMARY

An embodiment improves over the arrangements discussed in the foregoing.

An embodiment relates to a corresponding computer-implemented method as well as a related computer-program product, loadable in the memory of at least one computer, and including software-code portions for performing the steps of an embodiment of a method when the product is run on a computer. As used herein, reference to such a computer-program product is intended to be equivalent to reference to a computer-readable medium containing instructions for controlling a computer system to coordinate the performance of an embodiment of a method. Reference to “at least one computer” is evidently intended to highlight the possibility for an embodiment to be implemented in a distributed/modular fashion.

In various embodiments, bio signals received from a remote health-monitoring device may be used to tag a multimedia content with an additional set of personal metadata.

In various embodiments, such personal metadata may convey information, possibly confidential, about personal emotional and health status. In various embodiments, having to deal with “sensitive” information, personal data may be encrypted and a corresponding key be distributed to a limited set of trusted people.

In various embodiments, an encoder may be coupled with a specific remote health-monitoring device that will get the information from a particular person. In various embodiments, the output of such an encoder may be a conventional multimedia content with an additional set of metadata attached to the header of the content file.

In various embodiments, a function may extract a tag from the health/emotional status as a semantic meaning that can be perceived by an end-user (e.g., “sad”, “angry”, “very good health”, and so on).

Various embodiments may be based on the recognition that one of the main challenges of semantic retrieval systems is to exploit human-machine interaction through which end users tag their own multimedia content with labels and semantic references. End-users usually do not perform this task because they may consider it to be time consuming, boring, or annoying.

In that respect, it has been noted that, in the area of biomedical engineering, new breakthrough solutions in the field of health remote monitoring are being contemplated such as, e.g., wearable, comfortable, body-gateway devices able to measure biological and physiological indexes. Once part of the life of ordinary people (especially in an aging society), exploitation of the indexes made available by such devices may be considered also for other daily human activities.

For example, it might be possible to exploit information derived from speech analysis, breathing analysis, electrocardiogram analysis, steps analysis, altitude analysis, blood-pressure analysis, and others, in order to create labels with the aim of tagging multimedia (MM) contents.

Various embodiments aim at detecting and deducing a person's status while capturing additional multimedia content: the output may be an additional type of tag that is just attached to the same content.

Various embodiments may be related to the application of semantic retrieval through text key-words query. In various embodiments, an encoder may in fact produce just an additional semantic tag.

Various embodiments may be focused on a module in a complete architecture, with the aim of analyzing and processing bio indexes to detect an emotional/physiological state. In various embodiments, processing these bio-signals may lead to more sophisticated tags in the place of pure samples of the bio signal value over time.

In various embodiments, such a module may be able to detect emotional states such as fear, anxiousness, and relaxedness; changes in such state(s) may be detected and correlated to events that trigger such changes.

This may happen especially in connection with events where a sudden change in the external environment and conditions leads to a change in the morphological-parameter profile of the person, e.g.,—just by way of example—the heartbeat.

In various embodiments, a processing architecture may deliver information coming from the remote health-monitoring device; then, the type of tags produced may be processed at different levels of semantic abstraction though relying on the same system architecture; finally, in certain embodiments, an encoder may attach a tag to a multimedia content, with this tag providing personal information of a particular person who is, in general, the owner of the photo/video content or the owner of the device that produced the MM content.

It has been noted that the social implications and the impact of certain embodiments may be very high, especially if related to the “aggregated” profile of a whole community. For example, events such as a goal being scored in a stadium, an event on TV, a song in a concert, thunder during a storm, and other events being experienced, may be associated with multimedia contents that are possibly able to convey additional information about the subject portrayed in a picture or video, thus pursuing the aim of making such a subject feel “connected”.

Various embodiments thus make it possible to exploit and link multimedia contents to more personal information related to the “owner” of such multimedia contents.

In various embodiments this may result from merging contributions from “body gateway” devices for health monitoring and multimedia.

Various embodiments may relate to a system architecture that interfaces a multimedia content generator with a RHMS (Remote Health Monitoring Solution), with the aim to extract, process, and then attach a broad set of additional tags that includes, but is not limited to, Bio-status, emotional status, GPS, text label, and ID user, to the header of a multimedia content.

### BRIEF DESCRIPTION OF THE DRAWINGS

Various embodiments will now be described, by way of example only, with reference to the annexed figures, in which:

FIG. 1, including two portions designated 1a and 1b, respectively, is representative of various steps in embodiments;

FIG. 2 is representative of an embodiment a semantic encoder;

FIG. 3 shows different types of connections in various embodiments;

FIGS. 4 and 5 are representative of encoder and decoder embodiments, respectively;

FIGS. 6 and 7, with FIG. 7 including two portions designated 7a and 7b, respectively, are representative of encryption and decryption steps in embodiments;

FIGS. 8 to 10 are representative of various steps in embodiments.

### DETAILED DESCRIPTION

Illustrated in the following description are various specific details aimed at an in-depth understanding of the embodiments. The embodiments may be obtained without one or more specific details, or through other methods, components, materials, etc. In other cases, known structures, materials or operations are not shown or described in detail to avoid obscuring the various aspects of the embodiments. Reference to “an embodiment” in this description indicates that a particular configuration, structure, or characteristic described regarding the embodiment is included in at least one embodiment. Hence, expressions such as “in an embodiment”, possibly present in various parts of this description do not necessarily refer to the same embodiment. Furthermore, particular configurations, structures, or characteristics may be combined in any suitable manner in one or more embodiments.

References herein are used for facilitating the reader's understanding, and, thus, they do not define the scope of protection or the range of the embodiments.

This disclosure relates, by way of example, to an encoder-architecture system designed for an automatic metadata-tagging method to be used in association with any device able to produce multimedia content, including digital cameras or microphones

In the block diagrams of the figures, blocks may be either information data, an information item, a metadata item, a data string, or logic-module blocks such as an engine, a logic block, an algorithm, and so on.

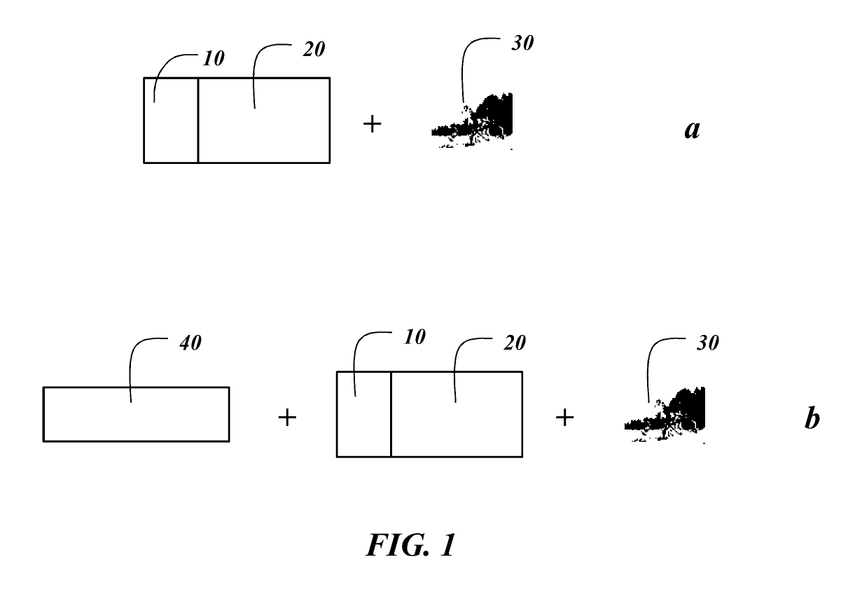

The two portions of FIG. 1, designated 1a and 1b, respectively, compare a conventional approach (FIG. 1a) and an embodiment of a approach newly proposed herein (FIG. 1 b), to produce multimedia contents from raw data and metadata. In certain embodiments, this result may be achieved by using an encoder.

With reference to FIG. 1a, the output of the encoder may include an exif-tags block 10 (where exif stands for “exchangeable image file format”), a metadata block 20, and an image (raw data) 30; exif-tags are a specification for an image file format used by the majority of digital-camera brands. Metadata content may include, for example, size and filename information.

In the case of FIG. 1 b, a type of metadata format is produced including additional metadata, indicated also as personal metadata.

In certain embodiments, the output of the encoder in FIG. 1 b may also include a personal metadata block 40. In various embodiments, the personal metadata 40 may be encrypted.

In various embodiments, the encoder of FIG. 1b may rely on a full architecture that may also produce personal metadata as described in the following.

In various embodiments, the encoder architecture may tag the multimedia content with health/emotional-status information. In various embodiments, the encoder architecture is able to manage multiple semantic tags in order to enrich the capability and efficiency of a semantic retrieval system and increase the user experience in a social-network scenario.

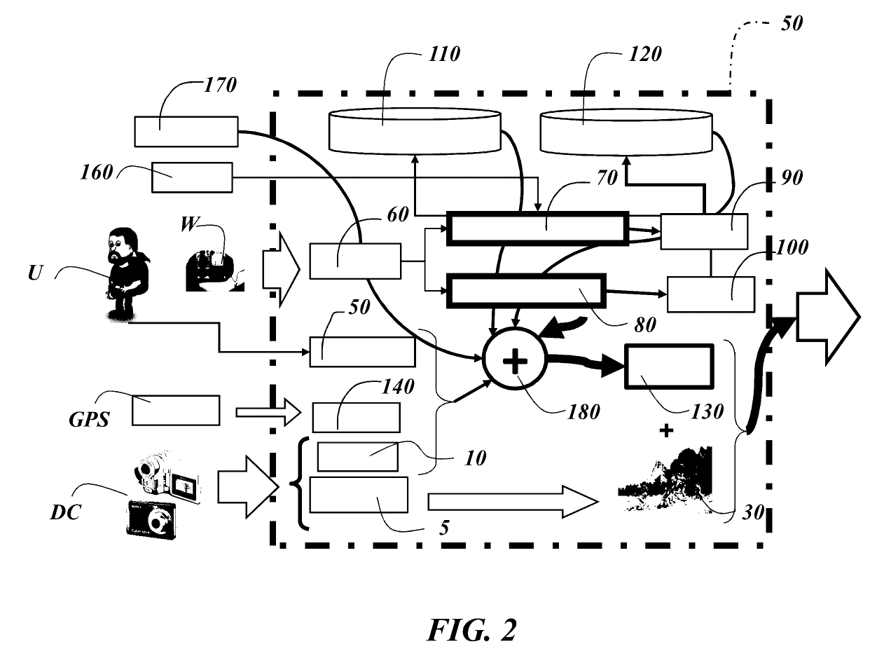

An exemplary architecture that represents a possible flow of signals is represented in FIG. 2. There, a user U is assumed to be equipped with a wearable monitor device W that measures biological and physiological indexes of the user U. The wearable monitor device W produces a set of bio indexes 60. These indexes 60 are made available as input for a “feel” function 70 and a “health” function 80. The output of the feel function 70 is “feeling status” information 90, and the output of the health function 80 is “health status” information 100.

Reference 50 indicates as a whole the exemplary semantic encoder architecture.

There, digital cameras or digital-video cameras DC may produce exif-tags 10 for each picture 5.

In certain embodiments, a GPS module may produce location information 140. For each user U, a block 50 may produce content ID related to that specific user U. Speech information 160 may also be used as an input parameter for the feel function 70.

A block 110 may include, e.g., a list of health categories, avatar and owner portrait, while a block 120 may include a list of feeling categories in addition to avatar and owner portrait. An additional text label 170 may be added.

All these information items may be merged in a block 180 in order to produce new additional tags 130 to be linked, e.g., to a picture 30.

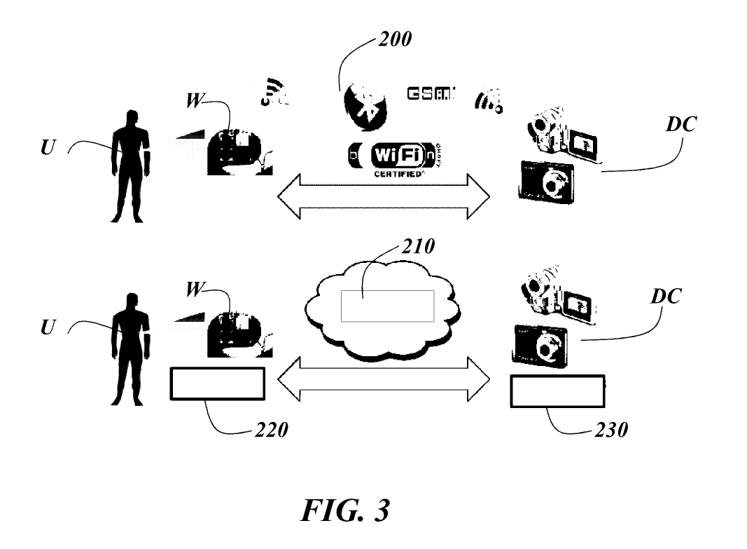

FIG. 3 is exemplary of a remote health-monitoring solution (RHMS) communicating with the digital cameras DC through a connection.

There, users U equipped with a wearable monitoring device W may be coupled to the digital cameras DC in several ways. Such connection has the purpose of coupling two devices (W and DC) and may, e.g., be wired/wireless; connection may be achieved by known means (e.g., through specific types of radio links), thus making it unnecessary to provide a detailed description herein.

Examples of connections may be:
short range connection 200 (Wireless, Bluetooth, Infrared, Radio Frequency), and
long range connection 210 through any type of wireless standard for data or voice communication (GPS, UMTS, EDGE, just as is the case with the Internet through two IP addresses 220 and 230).

In various embodiments, two generic devices may be provided with IP addresses. So, a multimedia-content generator and a remote health-monitoring device may be coupled once they are provided with IP addresses from any two locations in the world.

In various embodiments, the coupling mechanism considered herein may include the possibility of coupling a multimedia content not necessarily to a multimedia-content producer, but to any possible device that provides other metadata, i.e., bio metadata.

Thus, in various embodiments, a semantic encoder may receive as an input multimedia content from a multimedia-content generator, along with biosignals related to a person located elsewhere, even though not directly related to a same social event.

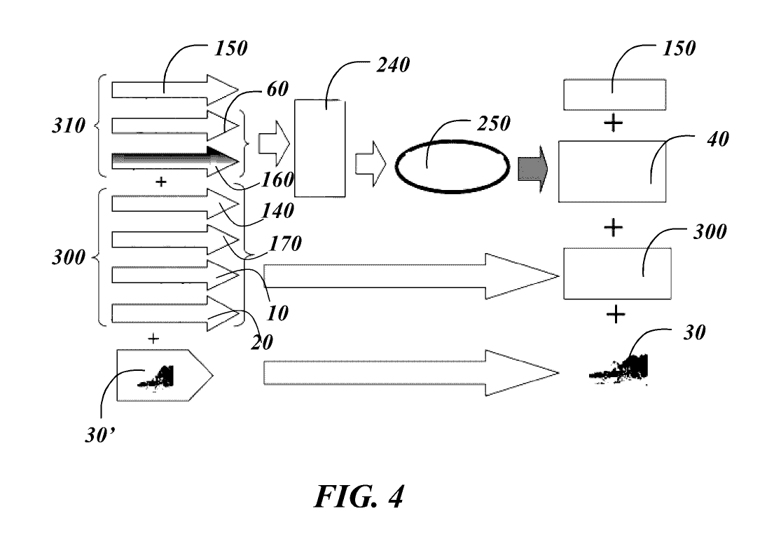

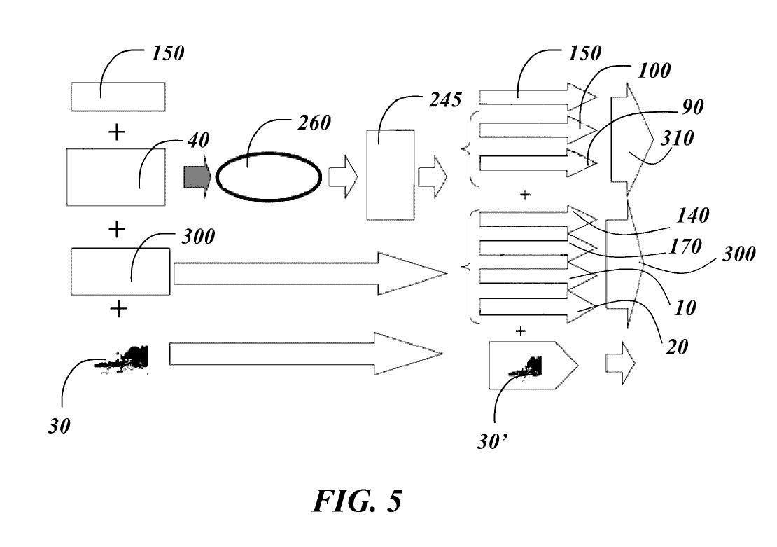

FIGS. 4 and 5 are representative of examples of managing metadata (encoder vs. decoder).

In the exemplary embodiment considered, all the tags are managed at the encoder side, see FIG. 4.

In this example, the “traditional” metadata 300, related, e.g., to a picture 30′, may include GPS data (or location information) 140, a text label 170, exif-tags 10, and basic metadata 20 (like size, filename, etc.).

In this example, the “new” additional metadata 310 may include ID content 150, bio-data 60, and speech-tone information 160.

In various embodiments, the latest information of the additional metadata 310 may be processed by a semantic processing block 240, and then encrypted via an encryption block 250 in order to produce personal metadata 40.

In that way, the information linked to the picture 30 is enriched with personal metadata 40.

An exemplary dual scheme at the decoder side is shown in FIG. 5. There, the personal metadata 40 are decrypted via a decryption block 260, and the results are parsed in a semantic parsing block 245.

The semantic processor block 240 (FIG. 4) matches logically the semantic parsing in the decoder.

In various embodiments, the tags that the end user is able to read may be accessed through a text/tag query for retrieval purposes.

For example, the bio-data input to the encoder side may become, e.g., a health-status-information item at the decoder side, and such health-status information may become metadata that is used to tag the content; this may then either be readable by the end user or be used for queries in multimedia-content search sessions.

In various embodiments, the metadata information that is considered confidential by the owner may be optionally hidden through encryption keys that are distributed only by the owner of this information.

In various embodiments, the confidential information may be associated with an ID producer that is not necessarily the owner of the content, or the owner of the camera device that shot the multimedia content.

In various embodiments, the ID in question may be an ID that links the information coming from the Remote Health Monitoring Solution, which may be coupled to the camera via, e.g., a network connection (of any known type). For instance, coupling may be via devices that are at “Bluetooth” short range, or, optionally, to any Remote Health Monitoring Solution device that is reachable.

In various embodiments, it may be possible to attach the metadata coming from the Remote Health Monitoring Solution of a close friend in the USA while taking a picture in Europe.

In various embodiments, the exemplary architecture considered may be fully determined by the access to the information available through the portable health-monitoring gateway device.

In various embodiments, the metadata that are referred to information that the user wants to hide may need a data-encryption mechanism and a digital signature. In various embodiments, while the encryption mechanism may hide the very value of the tags, the digital signature may ensure that these values are associated with an ID producer. In that case, the ID producer may be referred to the producer of the bio signals linked to the Remote Health Monitoring Solution.

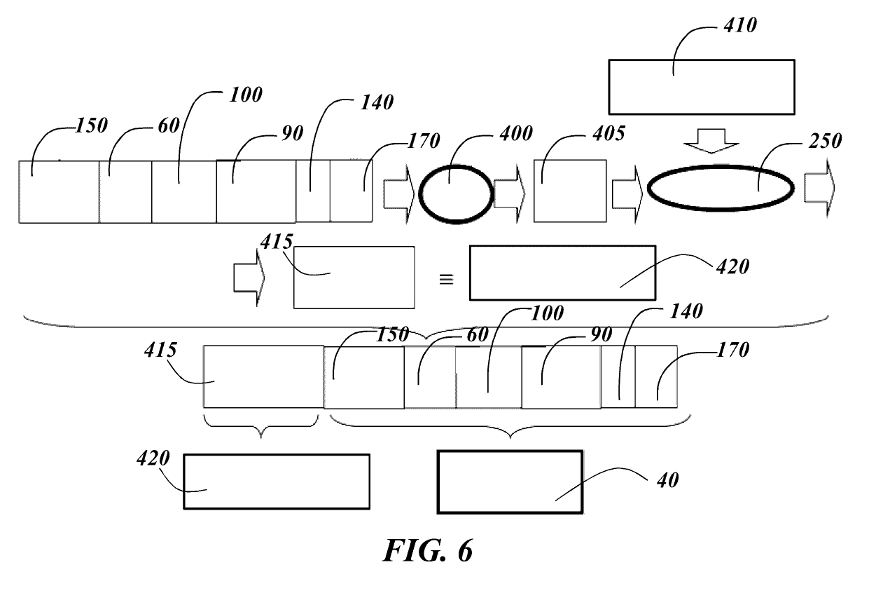

FIG. 6 is schematically representative of an embodiment of a kind of “author” digital-signature process.

In such an exemplary embodiment, a vector of information including, e.g., ID content 150, bio-index 60, health status 100, emotional status 90, geo-tags or location information 140, and text label 170 may be made available as an input to a hash block 400. Then the output of the hash block 400 may be sent to a digest block 405, and subsequently to an encryption step 250. The encryption step 250 may also receive an encryption key1 410 in order to produce hash-encrypted information 415 and a digital signature 420.

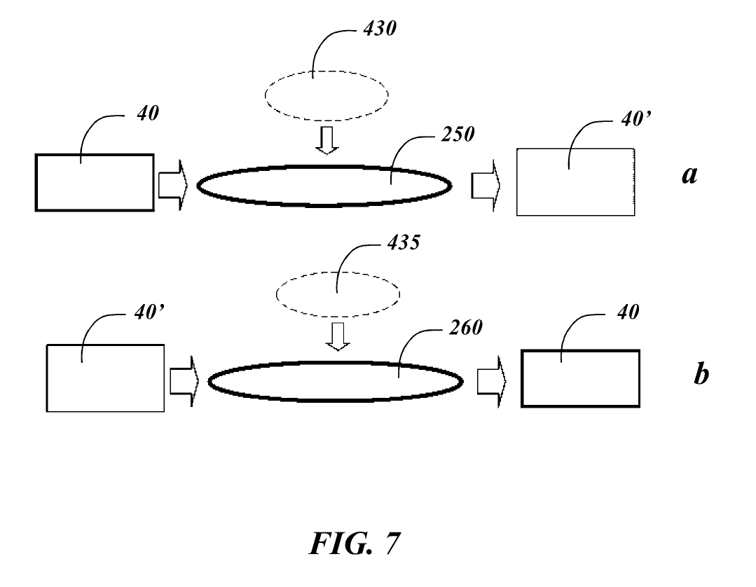

Exemplary encryption and decryption processes at the producer and the consumer sides are illustrated in FIG. 7a and FIG. 7b, respectively.

In such exemplary embodiments, the personal metadata 40 may be encrypted in a step 250 by using a private key2 430 in order to obtain an encrypted personal metadata 40′. At the producer side, the encrypted metadata 40′ may be decrypted in a step 260 using a public key2 435 for obtaining the original metadata 40.

While the private key 430 may be used to encrypt (hide) the personal metadata at the producer side, a public key 435 may be used to decrypt the personal metadata at the consumer side. The public key 435 may be distributed by a secret channel only to the people that are trusted by the producer.

In various embodiments, an exemplary Bio-Status function B(t) may be representative of the status of a whole set of bio signals b1(t), b2(t) bi(t), . . . bN(t), where N is the number of signals detected by the Remote Health Monitoring Solution system; the Bio-Status B(t) function may be, e.g., a vector function whose elements are represented by N scalar functions.

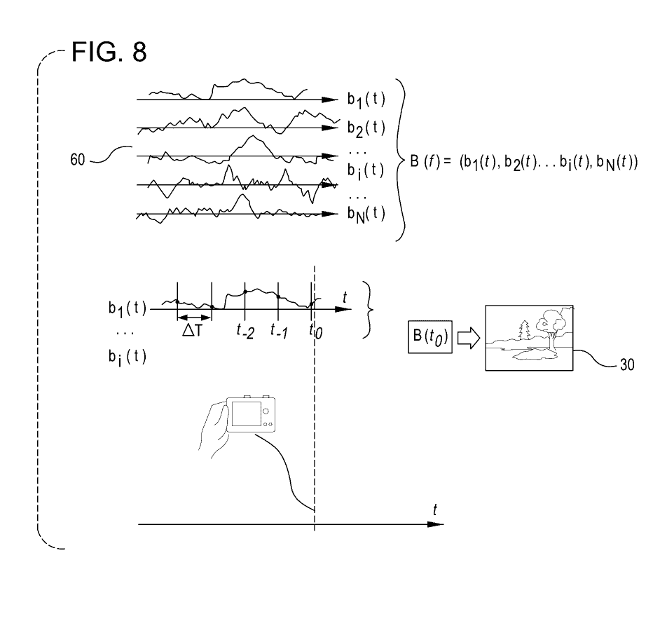

In that respect, FIG. 8 is schematically representative of an embodiment of a chain of computation steps performed on the bio signals 60. The exemplary chain considered herein describes a possible way of defining bio-tags and associating such bio-tags to a multimedia content.

In fact, because of low-power constraints, and, possibly, a semantic relevance of the bio-signals to an event, in various embodiments, a Bio-Status function B(t) of the RMHS user may be sampled at time intervals ΔT; after that the Bio-Status function B(t) may be attached to the multimedia content generated by the multimedia-content generator coupled with the RMHS generated device.

In various embodiments, a natural and intuitive matching may rely on the principle of temporal consistency, i.e., the multimedia content generated at time ti may be associated with the Bio-Status at the same time ti.

In various embodiments, the association between content generation and Bio-Status may be rendered temporally consistent, thus the ΔT may not need to be too short (microseconds) or too long (hours).

Possibly, in various embodiments, a value for ΔT may be (pre)set at a default value by the setup configuration of the device or through manual configuration of the device itself.

The possibility also exists of differentiating the temporal interval ΔT for each bio index 60 and along the timeline.

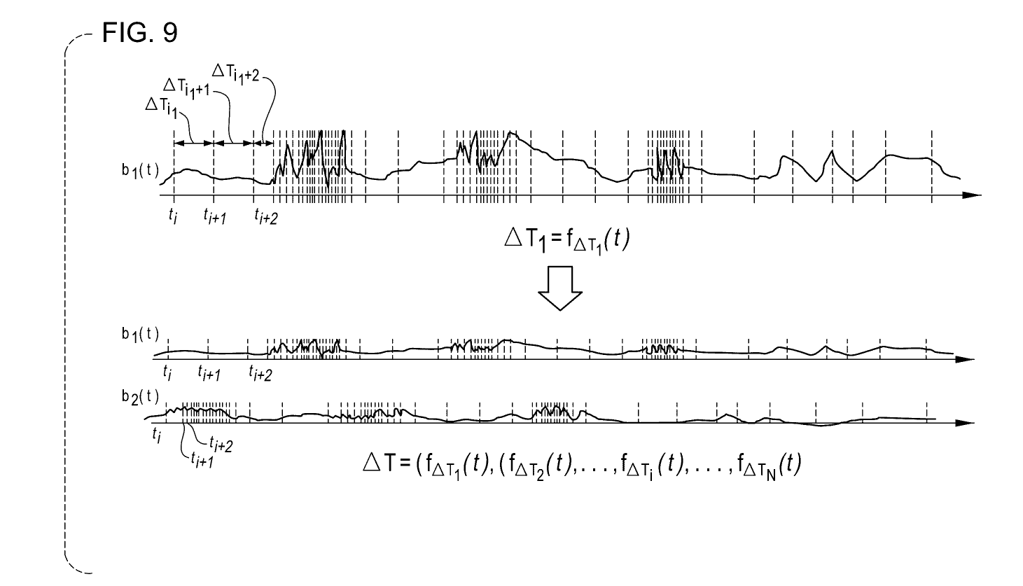

FIG. 9 is illustrative of an exemplary embodiment where the sampling interval is a function of time. A goal may be optimizing the use of resources when the bio activity is steady for a certain period of time, or becomes very variable.

This feature may be expressed by the introduction of a vector function ΔT, as indicated in equation 1.

ΔT=(fΔT1(t),fΔT2(t), . . . , fΔTi(t), . . . , fΔTN(t)) (1)

In certain embodiments, the possibility may exist of storing the whole bio history of the RMHS for each bio index in a Bio Status memory storage MBS. In that case, a buffer of values stored in the matrix of bio-index values MBS may be made available to be interrogated—should the need arise—when the multimedia content is generated. Additionally, in various embodiments, the possibility may exist of compressing the history in any compressed format and to access the memory storage MBS with any type of algorithm in the compressed domain and retrieve the relative bio index to be used as metadata for the multimedia-generated content, or other upper-layer applications.

Moreover, in certain embodiments, the possibility may exist of associating multimedia content with the bio-status by neglecting a too-obvious temporal correspondence. Common sense does in fact suggest that if a content is generated at a generic time t, then the association may be based on a temporal correspondence with the Bio Status sampled at the time t0 where t is: t0≦t<t1. However, in certain embodiments, it is possible to match the content generated at the time tx with the Bio Status sampled at ti where x<<i or i<<x.

Therefore it may be possible to attach the bio-status to a multimedia content while keeping a temporal distance from the multimedia-content generation.

In various embodiments, the bio data may be sampled and the bio-status attached to the multimedia content. In various embodiments, the additional tags may be generated from the bio indexes and put in the Semantic Encoder to produce more sophisticated tags to be used by the system architecture as metadata for the multimedia content.

An exemplary process of extracting bio-status information from the physiological signals will now be described.

In that respect, it has been noted that the human body is an excellent source of information. By recording and analyzing several physiological signals, it is possible to assess valuable data regarding the current health status of the user. Each signal conveys a different type of information, because it is based on a different physiological phenomenon: the ECG records the electrical activity of the heart, the PPG optically records blood perfusion, a thermometer can record variations in the skin temperature, and a microphone can record the audio signal of the heart beating.

By processing any such signal, a specific physiological index may be obtained, e.g., as a “synthetic value”, e.g., heart rate, breathing rate, temperature, etc. By combining all these different physiological indexes, the current status of the user can be estimated. A high heart rate may mean that the user was probably tired from a long walk, a high body temperature may mean that it was a really hot day—information that can greatly enhance the completeness of the data encoded with the multimedia file.

Finally, these physiological indexes can be used to estimate the feeling of the person, making it possible to understand, e.g., if the user is excited, angry, sleepy, and so on.

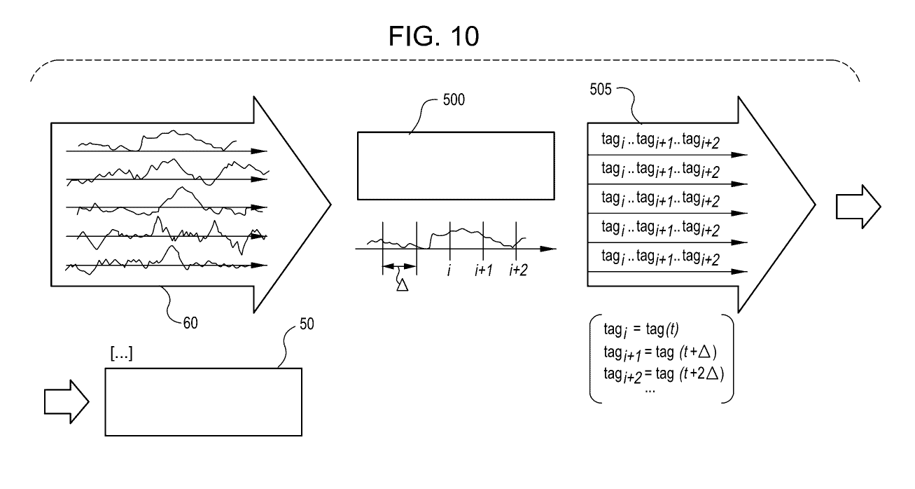

FIG. 10 is representative of an exemplary way of extracting in a step 500 bio data from the bio signals 60, which are analog signals, to subsequently generate tags 505. These tags 505 may be sent as an input to the semantic encoder 50.

For instance, tagᵢ=tag(t), tagᵢ₊₁=tag(t+Δ), tagᵢ₊₂=tag(t+2Δ).

The approach adopted in the encoder of this exemplary embodiment may be represented by the following features.
Health status (process module): the bio-signals are processed to retrieve or infer the end-user health status by a Fhealth function (Eq. 2). The Fhealth function may operate as follows:

Fhealth=f(B1,B2, . . . Bn) (2)

where Bi represents any bio-index that may be available from the RHMS or speech mood detection.

The output of the Fhealth function may include:

a) the use of a set of pre-defined health statuses such as icons, logos, different-type background colors, or color saturation/manipulation for the same photo (for example, the content-owner portrait), that aim to represent a limited number of health statuses;

b) the use of the above representation to indicate to the end-user viewer what was the health status at the moment of the multimedia-content capture.
Emotional status (process module): the correlation of the bio-signals with a speech-recognition mood engine to retrieve the available analog signals by any type of wired/wifi connection (usually Bluetooth) to infer the end-user emotional status by a Ffeel function (Eq. 3).

Ffeel=f(B1,B2, . . . Bi, . . . Bn) (3)

The output of the Ffeel function may include:

a) the use of a set of pre-defined emotional statuses such as icons, logos, different-type background colors, or color saturation/manipulation for the same photo, that aim to represent a limited number of emotional statuses;

b) the use of the above representation to indicate to the end-user viewer what was the emotional status at the moment of the multimedia-content capture.
To include health status as a semantic tag in the multimedia content.
To include emotional status as a semantic tag in the multimedia content.
To include the GeoTags (the place at the moment of the capture).
To include the content-owner identification of the multimedia content by any type of information that is univocally linked to the owner. Such form of identification includes any type of multimedia or textual tag such as a logo, icon, little digital portrait, a text file, a voice tag, a string ID, or a personal ID document.
To merge the GEO tags with the exif-tags metadata (a popular metadata standard used for all digital cameras).
To include specific text label indicated through:

a) Pre-defined set of labels inserted through a dedicated human machine interface;

b) Manually set label through keyboards inputs, or large-vocabulary automated speech-recognition engine, or other types of dedicated human-machine interface
A mechanism to protect the sensitive information by splitting the metadata between the GEO, exif-tags, and the other traditional tags with the personal metadata information. GEO location can, at preference of the user, be included in the set of personal/confidential metadata information. Then the confidential information needs a secure Hash algorithm to encrypt the tags that the user wants to hide.

The proposed architecture may be detectable by any end-user without any reverse engineering:
at the decoder side: in any retrieval system because multimedia contents may be retrievable through new specific tags such as bio-index tags, health status, or emotion status. This kind of encoder may actually permit a new type of semantic retrieval: emotion/health status retrieval (the multimedia content is being retrieved by querying an emotion/health status); and
at the encoder side: the users may wear and couple (e.g., via wireless) the portable gateway to the camera.

Various embodiments may be applied advantageously to the following areas:
on the decoder side, all systems adapted to perform even very simple forms of information retrieval, in particular “smart” Set Top Boxes, since retrieval is performed through tags;
on the encoder side, any device able to produce multimedia content, including digital cameras or microphones, where new tags may be added to conventional metadata;
portable body gateways; and
related software applications.

Without prejudice to the underlying principles of the disclosure, the details and embodiments may vary, even significantly, with respect to what has been described herein by way of non-limiting example only.

From the foregoing it will be appreciated that, although specific embodiments have been described herein for purposes of illustration, various modifications may be made without deviating from the spirit and scope of the disclosure. Furthermore, where an alternative is disclosed for a particular embodiment, this alternative may also apply to other embodiments even if not specifically stated.

### REFERENCES

[1] K. H. Kim, S. W. Bang, S. R. Kim, “Emotion recognition system using short-term monitoring of physiological signals”, Medical & Biological Engineering & Computing 2004, Vol. 42.
[2] J. Wagner, J. Kim, E. Andrè, “From physiological signals to emotions”, Multimedia and Expo, 2005. ICME 2005. IEEE International Conference on.
[3] H. S. Goldstein, R. Eldberg, C. F. Meier, L. Davis, “Relationship of resting blood pressure and heart rate to experienced anger and expressed anger”, Psychosomatic Medicine 50:321-329 (1988)
[4] D. Shapiro, D. Jamner, L. Goldstein, I. Delfino, “Striking a chord: Moods, blood pressure, and heart rate in everyday life”, Psychophysiology, Volume 38 Issue 2 Page 197-March 2001.

The above-listed references are incorporated by reference.

1.-11. (canceled)

12. An apparatus, comprising:
a first unit configured to generate a description of a subject in response to biological information on the subject; and
a second unit configured to associate the description with multimedia content that is related to the subject.

13. The apparatus of claim 12 wherein the description includes a mental state of the subject.

14. The apparatus of claim 12 wherein the description includes an emotional state of the subject.

15. The apparatus of claim 12 wherein the description includes a physiological state of the subject.

16. The apparatus of claim 12 wherein the biological information includes a physiological characteristic of the subject.

17. The apparatus of claim 12 wherein the biological information includes a vital sign of the subject.

18. The apparatus of claim 12 wherein the subject includes a human subject.

19. The apparatus of claim 12 wherein the multimedia content includes an image of the subject.

20. The apparatus of claim 12 wherein the second unit is configured to generate a file that includes a representation of the description and a representation of the multimedia content.

21. The apparatus of claim 12 wherein the second unit is configured:
to generate a heading that includes a representation of the description; and
to associate the heading with a representation of the multimedia content.

22. The apparatus of claim 12 wherein the first unit is configured to encrypt at least a portion of the description.

23. The apparatus of claim 12 wherein the first unit is configured to receive the biological information from a monitor coupled to the subject.

24. The apparatus of claim 12, further comprising a monitor configured to generate the biological information in response to a signal from the subject.

25. The apparatus of claim 12, further comprising a third unit configured to generate the multimedia content.

26. A system, comprising:
a first integrated circuit including
a first unit configured to generate a description of a subject in response to biological information on the subject, and
a second unit configured to associate the description with multimedia content that is related to the subject; and
a second integrated circuit coupled to the first integrated circuit.

27. The system of claim 26 wherein one of the first and second integrated circuits includes a controller.

28. The system of claim 26 wherein the first and second integrated circuits are disposed on a same die.

29. The system of claim 26 wherein the first and second integrated circuits are disposed on different dies.

30. A method, comprising:
generating a description of a subject in response to biological data related to the subject; and
associating the description with multimedia content that is related to the subject.

31. The method of claim 30 wherein the biological data includes a signal from the subject.

32. The method of claim 30 wherein associating includes:
generating a heading that includes a representation of the description and of other metadata; and
associating the heading with a representation of the multimedia content.

33. The method of claim 30, further comprising:
monitoring the subject; and
generating the biological data in response to the monitoring.

34. The method of claim 30, further comprising:
generating the biological data; and
generating the multimedia content.

35. A tangible computer-readable medium storing instructions that, when executed by at least one computing apparatus, cause the computing apparatus, or another apparatus controlled by the computing apparatus:
to generate a description of a subject in response to biological data related to the subject; and
to associate the description with multimedia content that is related to the subject.

36.-40. (canceled)
